SEQUENCE LISTING (28 sequences)

SEQ ID NO: 1 - DNA, 20 bp, Synthetic construct
  source 1..20: mol_type = other DNA
aaaaaccccccccccccccc

SEQ ID NO: 2 - DNA, 20 bp, Synthetic construct
  source 1..20: mol_type = other DNA
tttttggggggggggggggg

SEQ ID NO: 3 - DNA, 20 bp, Synthetic construct
  source 1..20: mol_type = other DNA
gggggttttttttttttttt

SEQ ID NO: 4 - DNA, 20 bp, Synthetic construct
  source 1..20: mol_type = other DNA
cccccaaaaaaaaaaaaaaa

SEQ ID NO: 5 - DNA, 20 bp, Synthetic construct
  source 1..20: mol_type = other DNA
cccccccccccccccggggg

SEQ ID NO: 6 - DNA, 20 bp, Synthetic construct
  source 1..20: mol_type = other DNA
gggggggggggggggccccc

SEQ ID NO: 7 - DNA, 20 bp, Synthetic construct
  source 1..20: mol_type = other DNA
tttttttttttttttttttt

SEQ ID NO: 8 - DNA, 20 bp, Synthetic construct
  source 1..20: mol_type = other DNA
aaaaaaaaaaaaaaaaaaaa

SEQ ID NO: 9 - DNA, 40 bp, Synthetic construct
  source 1..40: mol_type = other DNA
aaaaacccccccccccccccgggggttttttttttttttt

SEQ ID NO: 10 - DNA, 40 bp, Synthetic construct
  source 1..40: mol_type = other DNA
gggggggggggggggcccccaaaaaaaaaaaaaaaaaaaa

SEQ ID NO: 11 - DNA, 40 bp, Synthetic construct
  source 1..40: mol_type = other DNA
cccccccccccccccgggggtttttttttttttttttttt

SEQ ID NO: 12 - DNA, 40 bp, Synthetic construct
  source 1..40: mol_type = other DNA
tttttgggggggggggggggcccccaaaaaaaaaaaaaaa

SEQ ID NO: 13 - DNA, 22 bp, Synthetic construct
  source 1..22: mol_type = other DNA
aaaacccccccccccccccggg

SEQ ID NO: 14 - DNA, 23 bp, Synthetic construct
  source 1..23: mol_type = other DNA
ttttgggggggggggggggcccc

SEQ ID NO: 15 - DNA, 19 bp, Synthetic construct
  source 1..19: mol_type = other DNA
aaaattttttttttttttt

SEQ ID NO: 16 - DNA, 19 bp, Synthetic construct
  source 1..19: mol_type = other DNA
ttttaaaaaaaaaaaaaaa

SEQ ID NO: 17 - DNA, 41 bp, Synthetic construct
  source 1..41: mol_type = other DNA
cccccccccccccccggggaaaatttttttttttttttggg

SEQ ID NO: 18 - DNA, 42 bp, Synthetic construct
  source 1..42: mol_type = other DNA
gggggggggggggggccccttttaaaaaaaaaaaaaaacccc

SEQ ID NO: 19 - DNA, 41 bp, Synthetic construct
  source 1..41: mol_type = other DNA
aaaacccccccccccccccgggaaaattttttttttttttt

SEQ ID NO: 20 - DNA, 42 bp, Synthetic construct; identical to SEQ ID NO: 18
  source 1..42: mol_type = other DNA

SEQ ID NO: 21 - DNA, 50 bp, Synthetic construct
  source 1..50: mol_type = other DNA
aaaagagtcnnnnnaaaacccccccccccccccggggnnnnngactcggg

SEQ ID NO: 22 - DNA, 51 bp, Synthetic construct
  source 1..51: mol_type = other DNA
ttttctcagnnnnnttttgggggggggggggggccccnnnnnctgagcccc

SEQ ID NO: 23-24 - skipped (2 entries)

SEQ ID NO: 25 - DNA, 23 bp, Synthetic construct
  source 1..23: mol_type = other DNA
aaaacccccccccccccccgggg

SEQ ID NO: 26 - DNA, 23 bp, Synthetic construct
  source 1..23: mol_type = other DNA
ttttgggggggggggggggcccc

SEQ ID NO: 27-28 - skipped (2 entries)

(19) United States
(12) Patent Application Publication
(10) Pub. No.: US 2025/0101482 A1
(43) Pub. Date: Mar. 27, 2025

(54) HETERODUPLEX THEROMSTABLE LIGATION ASSEMBLY (HTLA) AND/OR CYCLIC HETERODUPLEX THERMOSTABLE LIGATION ASSEMBLY (CHTLA) FOR GENERATING DOUBLE-STRANDED DNA FRAGMENTS WITH SINGLE-STRANDED STICKY ENDS

(71) Applicant: UNIVERSITY OF MARYLAND, BALTIMORE COUNTY, Baltimore, MD (US)

(72) Inventors: Charles J. BIEBERICH, Brookeville, MD (US); Xiang LI, Catonsville, MD (US)

(21) Appl. No.: 18/890,153

(22) Filed: Sep. 19, 2024

Related U.S. Application Data
(63) Continuation-in-part of application No. PCT/US2023/064977, filed on Mar. 27, 2023.
(60) Provisional application No. 63/323,542, filed on Mar. 25, 2022.

(51) Int. Cl. C12P 19/34

(52) U.S. Cl. CPC C12P 19/34

(57) ABSTRACT

The present invention relates to a process for generating single-stranded overhangs (sticky ends) of a user-defined length and sequence in a predominantly double-stranded DNA molecule, wherein the process is restriction endonuclease and DNA exonuclease activity-independent and wherein formed heteroduplex DNAs are joined by one or more ligations using a DNA ligase with an end result of generating double-stranded DNA fragments with single-stranded overhangs for joining and generating larger linear or covalently closed circular DNA molecules with the option of variable regions.

### CROSS-REFERENCE TO RELATED APPLICATION

This application is a continuation-in-part application and claims priority to International Patent Application No. PCT/US2023/064977 filed on 27 Mar. 2023, which claims priority to U.S. Provisional Patent Application No: 63/323,542, filed on Mar. 25, 2022, the contents of which are hereby incorporated by reference herein for all purposes.

### INCORPORATION BY REFERENCE

In compliance with 37 C.F.R. § 1.52(e)(8), the sequence information contained in electronic file name UMBC_40374_503_SequenceListing, size 23,385 bytes, created on: 19 Sep. 2024 using WIPO Sequence 2.3.0 is hereby incorporated herein by reference in its entirety.

### BACKGROUND OF THE INVENTION

### Technical Field

The present invention relates to a process for generating single-stranded overhangs (sticky ends) of a user-defined length and sequence in a predominantly double-stranded DNA molecule, wherein the process is restriction endonuclease and DNA exonuclease activity-independent and wherein formed heteroduplex DNAs are joined by one or more ligations using a DNA ligase with an end result of generating double-stranded DNA fragments with single-stranded overhangs for joining and generating larger linear or covalently closed circular DNA molecules with the option of variable regions.

### Related Art

In the prior art, assembling multiple DNA fragments in an ordered manner has been accomplished using restriction enzyme-based cloning, however the process is often severely limited by the availability of compatible and appropriately located restriction enzyme sites. All in vitro ordered assembly methods require complementary single-stranded DNA (sticky) cohesive ends to direct DNA fragment order. Methods are distinguished principally by the approach whereby sticky ends are generated and their length. For example, most Type II restriction endonucleases used in conventional DNA cloning recognize palindromic sequences and cleave phosphodiester bonds within the palindrome in double stranded DNA leaving staggered cohesive (sticky) ends leaving, at most, a five nucleotide-long overhang. To achieve ordered assembly, each DNA fragment to be joined must have unique sticky ends, generated, for example, by the enzymatic activity of different restriction endonucleases. In practice, the number of fragments that can be joined in this manner to yield a circular DNA in a plasmid vector is generally accepted to be fewer than ten (10). Type IIS restriction endonucleases recognize specific DNA sequences that may or may not be palindromic then cut at a precise distance away from the recognition site within any DNA sequence, but also yield short sticky ends of no more than five nucleotides and most often four nucleotides. For example, a Type IIS restriction endonuclease forms the basis of so-called Golden Gate Cloning. Given that there are 256 possible iterations of four nucleotide overhangs, theoretically, up to 128 DNA fragments can be joined in an ordered manner a single-pot Golden Gate reaction. However, given that DNA ligases accept and ligate some sticky ends with incompletely Watson-Crick base paired sequences, the practical limit is 24 fragments. Another prominent assembly technique termed Gibson Assembly is homology based and uses bacteriophage exonucleases, i.e., T5 and T7 exonucleases to generate partially single-stranded DNA molecules. However, using the Gibson Assembly is limited because the number of fragments that can be joined in an ordered array is generally considered to be fewer than 20.

In order to overcome the shortcomings of previous assembly technologies the present invention provides for more efficient, high-fidelity technologies for generating DNA assemblies which are not limited by the number of joined fragments.

### SUMMARY OF THE INVENTION

The present invention provides for an efficient DNA assembly process that generates ligation-ready single-stranded overhangs of user-defined sequence and length from one to thousands of nucleotides forming heteroduplex DNAs with 5′ or 3′ overhangs, that being, the creation of sticky-end DNA molecules by the process of the present invention referred to as a heteroduplex thermostable ligase assembly (HTLA) process that forms linear or closed circular DNA molecules from double-stranded or single-stranded DNA precursor molecules, and when such process is performed for more than one cycle by the process referred to as Cyclic Heteroduplex Thermostable Ligase Assembly (CHTLA) beginning with double-stranded DNA precursors, the formation of an admixture of linear DNA molecules with sticky ends, circular DNA molecules and/or blunt-ended linear DNA molecules, and when CHTLA is performed beginning with single-stranded precursors, the formation of exclusively linear DNA molecules with sticky ends and/or circular DNA molecules. Herein, we define a ‘heteroduplex’ DNA molecule as a mostly double-stranded molecule wherein the ‘top’ strand originates from one double-stranded or single stranded precursor DNA molecule, and the ‘bottom’ strand originates from a different double-stranded or single-stranded DNA molecule, having been joined by Watson-Crick base pairing in the process known as annealing of, or hybridization of, complementary DNA strands.

In one aspect the present invention provides for a method of forming Sticky-End Blocks (SEB) with 5′ or 3′ overhangs, the method comprising:
providing at least three precursor single-stranded, double stranded or an admixture thereof DNA fragments and designed to correctly assemble to generate a defined DNA sequence;
introducing the at least three precursor DNA fragments into a buffer medium;
applying heat at a melting temperature to cause melting of the at least three precursor DNA fragments; and
lowering the melting temperature for annealing in the presence of a thermostable ligase, thereby generating heteroduplex double-stranded DNA formed by base pairing of complementary regions that juxtaposes DNA ends that are then ligated thereby generating a new larger double-stranded DNA product with single stranded ends with 5′ or 3′ overhangs termed a sticky-end block (SEB), or in the case when the overhangs on the ends of the SEB are complementary, a covalently closed circular DNA.

The precursor DNA fragments as used herein are selected from the group of double-stranded DNA molecules, single-stranded DNA molecules, or an admixture of the two. Nucleotide bases within said DNA fragments can be native adenine, guanine, cytosine, thymine, or any chemically modified form thereof that can be incorporated into a DNA molecule by chemical synthesis or the action of an enzyme, i.e., a DNA polymerase, or that can be chemically or enzymatically caused to appear in a base or bases after synthesis.

The above-described method of forming Sticky-End Blocks (SEB) with 5′ or 3′ overhangs includes precursors DNA fragments and a thermostable DNA ligase enzyme in a buffer medium comprising a buffer to maintain pH, for example, Tris-HCL, MgCl₂, KCl, NAD, DTT, and Triton X-100 and at a pH of about 4.0 to about 10 and preferably from about 6 to 10 more preferably from about 7.5 to about 9. The melting temperature for can range from about 37° C. to 100° C., more preferably above 60° C. wherein the time frame for melting ranging from about 30 seconds to about 10 minutes, and more preferably from 1 to 5 minutes. The annealing is conducted by lowering temperature from 5° C. to 60° C. lower than the melting temperature, and more preferable from about 10° C. to 40° C. lower than the melting temperature and the time frame for such annealing step is from about 30 seconds to 10 mins and more preferably from about 4 mins to 6 mins.

Further, for the production of an admixture of SEBs and blunt-ended DNAs, the above method can employ double-stranded DNA precursors and repeated numerous times, for example from at least 2 to 12 or more times. Note that in each cycle beyond cycle 2, the blunt-ended products formed may also contribute to the formation of the desired SEBs.

In another aspect, the present invention provides for a method of forming Sticky-End Blocks (SEB) with 5′ or 3′ overhangs, the method comprising:
providing at least three precursor single-stranded, double stranded or an admixture thereof DNA fragments and designed to correctly assemble to generate a defined DNA sequence;
introducing the at least three precursor DNA fragments into a buffer medium at a pH of about 7.5 to about 9 and comprising Tris-HCL, MgCl₂, KCl, NAD, DTT, and Triton X-100;
applying heat at a temperature to cause melting of the at least three precursor DNA fragments, wherein the temperature for melting is determined by the size of the sequence and can range from about 37° C. to 100° C. with a heating time frame ranging from 30 seconds to 10 minutes; and
lowering the temperature for annealing in the presence of a thermostable ligase, wherein annealing is conducted at a temperature from 10° C. to 40° C. lower than the temperature for melting and for a time frame ranging from 4 mins to 10 mins thereby generating heteroduplex double-stranded DNA formed by base pairing of complementary regions with single-stranded ends thereby forming the sticky-end blocks with 5′ or 3′ overhangs.

Additionally, the precursor DNA fragments may comprise one or more random or variable nucleotides. Further, any precursor double-stranded DNA fragments that are not consumed in the above-described reaction can re-form upon annealing. If more than one cycle of heating, annealing, and ligation are performed, in addition to the new SEB products, new blunt ended products are also formed as discussed below.

Further, the buffering medium may include a single-stranded binding protein, enzymes such as a DNA helicase or topoisomerase, etc., one or more crowding agents, metal ions, detergents or other agents that promote DNA strand annealing.

In yet another aspect the present invention provides for a method of forming circular heteroduplex DNAs, the method comprising:
providing at least three precursor DNA fragments and designed to correctly form one or more SEBs that assemble to generate a defined covalently closed circular DNA sequence;
introducing the at least three precursor DNA fragments into a buffer medium at a pH of about 7.5 to about 9 and comprising Tris-HCL, MgCl₂, KCl, NAD, DTT, and Triton X-100;
applying heat at a temperature to cause melting of the at least three precursor DNA fragments, wherein the temperature for melting is determined by the size of the sequence and can range from about 60° C. to 100° C. with a heating time frame ranging from 30 seconds to 10 minutes;
lowering the temperature for annealing in the presence of a thermostable ligase, wherein annealing is conducted at a temperature from 5° C. to 40° C. lower than the temperature for melting and for a time frame ranging from 1 min to 10 mins thereby generating heteroduplex double-stranded DNA formed by base pairing of complementary regions with single stranded ends and thereby forming the sticky-end blocks with 5′ or 3′ overhangs; and
repeating the step of heating and annealing steps multiple times to form an admixture of covalently closed circular DNAs and blunt-ended linear DNA sequences.

Optionally, SEBs can be created in separate reactions, then combined to create a larger SEB or a covalently closed circular DNA (cccDNA). For example, if 20 DNA precursors are to be joined, five can be joined in Reaction 1, five in Reaction 2, five in Reaction 3, and five in Reaction 4. The products of the four Reactions can then be combined and joined by a further HTLA or CHTLA reaction, or by direct enzymatic ligation of the designed complementary sticky ends of the products of each the four Reactions, for example, using T4 DNA ligase or any other suitable DNA ligase at the appropriate temperature.

In another aspect, the desired SEB products of Reactions 1-4 may be purified, for example, by agarose gel purification, or any other means of separation, from precursor DNAs, then combined for an HTLA or CHTLA reaction to generate an SEB or an SEB and blunt-ended products comprised of all 20 precursor DNAs.

The precursor DNAs according to the present invention can range from about 20 nucleotides to thousands of nucleotides in length, and more preferably from about 200 to 10000 nucleotides for double-stranded precursors, and 30 to 200 for single-stranded precursors. The number of DNA precursor fragments will determine the size and length of SEBs formed and the length of the sticky ends on the ends of the SEBs prepared by the methods of the present invention.

Accordingly, the present invention provides nucleic acid ligation schemes that require temperature cycling such as cycling from, e.g., about 95° C. to a lower temperature of about 60° C. for one, two, three, or more cycles.

In yet a further aspect the present invention provides for a method of forming Sticky-End Blocks (SEB) with 5′ or 3′ overhangs, the method comprising:
providing at least three single-stranded precursor DNA molecules (oligonucleotides) and designed to correctly assemble to generate a defined DNA sequence;
introducing the at least three paired precursor DNAs into a buffer medium;
applying heat at a melting temperature to cause melting of the at least three paired precursor DNA fragments; and
lowering the melting temperature for annealing in the presence of a thermostable ligase, thereby generating heteroduplex double-stranded DNA formed by base pairing of complementary regions with single stranded ends thereby forming the sticky-end blocks with 5′ or 3′ overhangs.

The precursor DNA fragments according to the present invention comprise a unit of measurement designating the length of DNA ranges from about 0.1 kb to about 100 kb, and more preferably from about 0.5 kb to about 5 kb. The number of complementary paired precursor DNA fragments determines the size and length of SEBs prepared by the methods of the present invention.

Other features and advantages of the invention will be apparent from the following detailed description, drawings and claims.

### BRIEF DESCRIPTION OF FIGURES

FIG. 1 shows Heteroduplex Thermostable Ligation Assembly (HTLA) to generate a 100 base-long Sticky-End Block (SEB) with 10-base sticky ends from three 5′ phosphorylated double-stranded DNA precursors according to the present invention. Precursor A bears partial sequence identity with Precursor C, as indicated by the blue color (nucleotides 11-50 of the 100-base sequence). Precursor B bears complete sequence identity Precursor C as indicated by the purple color (nucleotides 51-90 of the 100-base sequence). Upon melting of the double-stranded precursors, the ‘top strands’ of Precursors A and B may anneal to the ‘bottom’ strand of Precursor C as shown, and vice versa (not shown). In the resulting heteroduplex, at the junction between the last nucleotide of Precursor A (base #50) and the first nucleotide of Precursor B (base #51), the so-called nick in the phosphodiester backbone is sealed by the enzymatic activity of a DNA ligase. The resulting SEB is a 100 base-long molecule wherein 80 Watson-Crick base pairs are formed, with 10 base-long 5′ sticky ends. Note that the ‘converse’ SEB with 3′ overhang stick ends also forms (not shown).

FIG. 2 shows Cyclic Heteroduplex Thermostable Ligation Assembly (CHTLA) to Generate Sticky-End Blocks (SEBs) according to the present invention. Note that this figure shows the generation of an SEB from four phosphorylated double-stranded DNA Precursors (i.e., PCR products generated using 5′-phosphorylated oligonucleotide primers), two mostly from DNA Region 1, and two mostly from DNA Region 2. The ‘top’ strands of each precursor are indicated by solid-colored lines, and ‘bottom’ strands are indicated by hashed colored lines. Note that self-annealing of the phosphorylated DNA Precursors (gray dashed arrow) simply regenerates the precursor molecules. The two SEBs that form, one with 5′ overhanding sticky ends and one with 3′ overhanging sticky ends encompassing all of DNA Region 1 and DNA Region 2 are shown. Although four are depicted here, SEBs can be generated by cyclic ligation from a theoretically unlimited number of PCR Precursors. Note that in every cycle beyond Cycle 1, blunt-ended blocks can also form as shown, encompassing most but not all of DNA Regions 1 and 2. These blunt-ended products can also serve as precursors to form SEBs in subsequent cycles.

FIG. 3 shows an example of an HTLA reaction with four double-stranded DNA precursors with nucleotide sequences shown. Note that the upper-case letters in the precursors indicate nucleotides that will constitute sticky ends in the product SEB, and lower-case letters indicated nucleotides that will be Watson-Crick base paired in the product SEB. Note that in the product SEBs, SEQ ID NO. 9 is generated by the ligation of SEQ ID NO. 1 and SEQ ID NO. 3. SEQ ID NO. 10 is generated by the ligation of SEQ ID NO. 6 and SEQ ID NO. 8. Likewise, SEQ ID NO. 11 is formed by the ligation of SEQ ID NO. 5 and SEQ ID NO. 7, and SEQ ID NO. 12 is generated by the ligation of SEQ ID NO. 2 and SEQ ID NO. 4.

FIG. 4 shows CHTLA to assemble a partial open reading frame (ORF) of the human gene SAP130. Four ˜550-bp double-stranded DNA precursors were joined in a 10-cycle CHTLA using HiFi Taq ligase (Lane 3) or Ampligase® (Lane 4) to generate a partial SAP130 ORF. Note complete conversion of ˜550-bp precursors (solid arrows) to 1.1-kb product (solid arrow) using HiFi Taq ligase.

FIG. 5 shows the products of CHTLA to assemble a complete ORF of the human gene SAP130. Ten double-stranded 450-750 bp DNA precursors (Lane 2, green arrows) were joined in a 10-cycle CHTLA reaction using HiFi Taq ligase to yield a 3.05 kb product (Lane 3, green arrow). CHTLA products digested with Xba I+Sal I (Lane 4) yielded an expected 2.6 kb product (orange arrow) confirming correct assembly. The 2.6 kb band was excised and cloned into pBluescriptSK-. Restriction digestion of DNA from 2/2 resultant colonies further confirmed correct assembly (data not shown).

FIG. 6A shows Heteroduplex thermostable ligase assembly (HTLA) according to the present invention; * indicates 5′-PO4.

FIG. 6B shows the gel image from a 5-precursor HTLA to generate the 3.0 kb ORF of human SAP130. The colored bars above the gel image shows the arrangement of overlapping and offset double stranded DNA precursors 1.6, 1.45, 1.25, 1.0, & 0.8 kilobase pairs in length. Lane 1, double stranded DNA precursors only; Lane 2, HTLA reaction products using Ampligase thermostable ligase; Lane 3, HTLA reaction products using HiFi Taq thermostable ligase; Lane 4 DNA marker. White arrow, SEB product.

FIG. 7 shows Cyclic Heteroduplex thermostable ligase assembly (CHTLA) according to the present invention and illustrates the results of a 1^{st} and 2^{nd} cycle.

FIG. 8 shows a strategy for performing HTLA with a mix of double-stranded and single-stranded precursors (oligonucleotides), and for incorporating oligonucleotides with randomized regions into SEBs according to the present invention. Note that four of the DNA precursors are double-stranded, and two are single-stranded oligonucleotides. In the oligonucleotides shown, N refers to any of the four canonical nucleotides (i.e., A, T, G, or C). Identical DNA sequences in the precursors are indicated by sameness of color.

FIG. 9A shows a strategy for creating HTLA Precursors with perfect blunt ends according to the present invention. Note that in ‘regular’ PCR, incomplete products are generated that may be one, two, or more nucleotides ‘short’, leaving an overhang. Since these products are unsuitable for HTLA, as they would leave gaps in the resulting heteroduplex DNAs (SEBs) that form the rectangle in FIG. 9A.

FIG. 9B shows that it is highly desirable to create ‘perfect’ blunt ended DNA precursors by employing a Type IIS restriction enzyme (i.e., MlyI) to ‘polish’ the ends of the PCR-generated DNA precursor prior its use in an HTLA or CHTLA reaction.

FIG. 10A shows construction of an 8.8 kb plasmid from four HTLA/CHTLA-generated SEBs using the strategy to produce an 8.8 kilobase pair (kb) circular DNA molecule by ligation of four SEBs created by HTLA or CHTLA. Note that one of the SEBs encompasses a plasmid vector for propagation of the DNA in bacteria.

FIG. 10B shows the generation of four SEBs by CHTLA: Lane 1, a 2.9 kb vector SEB was generated in a one-cycle HTLA reaction from 4 PCR precursors (1.2 kb, 1.7 kb, 1.9 kb and 1.0 kb). Three “insert” SEBs were generated in 10-cycle CHTLAs (95° C. for 1 minute, 60° C. for 5 minutes×10) as follows: SEB 1 (1.7kb final), from 0.35 kb, 1.35 kb, 0.95 kb, and 0.75 kb precursors; SEB 2 (2.5 kb final) from 1.6 kb, 0.9 kb, 1.7 kb, and 0.8 kb precursors; SEB 3 (1.7 kb final) from 0.6 kb, 0.6 kb, 1.1 kb and 1.1 kb precursors. The four independently generated SEBs were ligated at room temperature with T4 DNA ligase. SEB sticky ends=6 nucleotides. T4 ligation products were transformed into the bacterial strain DH5α. Correct clones identified by colony PCR were confirmed by restriction digestion (Xho I+Sac I) of mini-prepped DNA.

FIG. 11 shows a strategy to generate Sticky End Blocks with Randomized sequences near the End (SEB^{REs}) by introduction of randomizing DNA oligonucleotides on SEB ‘ends’ (just internal to sticky ends) according to the present invention. Note that four double-stranded precursors are shown (precursors I-IV) and two single-stranded precursors (Randomized Oligos I & II). One application of this strategy would be to create a DNA library wherein two regions were randomized or sequence limited. In the Randomized oligos, the L portion constitutes the ligatable portion of the sticky end, the R region has a randomized sequence, and the H region is homologous to either Precursor I or Precursor IV. Note also that the vector depicted is generated by HTLA to contain sticky ends that are complementary to the L regions of Randomizing oligos I and II. Ligation of the SEB^{RE} to the SEB vector would yield a closed circular DNA molecule with gaps in the randomizing region.

FIG. 12 shows a strategy to generate Sticky End Blocks with Randomized sequences Internally (SEB^{RIs}) by introduction of internal randomizing oligos according to the present invention. In this strategy, Precursor I would be generated by polymerase chain reaction using an oligonucleotide primer with a randomized sequence. Precursor III would have the wild type sequence in the region corresponding to the randomized region in Precursor I. SEB^{RI}S thus produced would have mismatches in the randomized region that would be repaired upon, for example, transformation into bacteria using the endogenous bacterial mismatch repair system.

FIG. 13A shows that the commercial circular plasmid pBluescript II (SK-) (2.961 kb) can be digested with restriction enzymes (REs) to generate four blunt-ended DNA precursors using Pvu II alone, or Eco RV+Xmn I (E+X).

FIG. 13B, Lane PvuII shows that Pvu II digestion produces two blunt-ended DNAs, ˜2.51 kb and ˜0.45 kb, and FIG. 13B Lane E+X shows that XmnI+PvuII digestion produces two blunt-ended DNAs, ˜1.95 kb and 1.01 kb. FIG. 13B shows the products of a 10-cycle CHTLA reaction with the four restriction enzyme digestion-generated blunt-ended DNA precursors shown in FIG. 13A, in which the thermostable DNA ligase was 9 Degree North ligase (9N). The products of that HTLA reaction are shown in FIG. 13B, in Lane 9N. A product with apparent size of ˜4.5 kb is clearly visible on the gel (white arrow), which is the expected position of a closed circular 3 kilobase pair DNA. The HLTA reaction products were subsequently transformed into E. coli, and plasmid DNA from resultant E. coli colonies that had taken up HTLA product DNA was restriction enzyme-digested with Eco RV+Xmn I.

FIG. 13C shows that 5/5 colonies arising from HTLA product transformation into E. coli yielded the expected DNA banding pattern for a correct assembly. Together, FIGS. 13A-C show that CHTLA of the present invention efficiently produces circular DNA.

FIG. 14 show a strategy to generate SEB with three DNA precursors using the HTLA system of the present invention and results of one cycle. Note uppercase lettering (A, B, C) denotes ‘top’ strands of DNA precursors and lowercase lettering (a, b, c) denotes ‘bottom’ strands. The two SEB products, one with 5′ overhanging sticky ends, and the other with 3′ overhanging sticky ends, are shown.

FIG. 15A shows a strategy to generate SEB with three DNA precursors using the HTLA system of the present invention. Note uppercase lettering (A, B, C) denotes ‘top’ strands of DNA precursors and lowercase lettering (a, b, c) denotes ‘bottom’ strands.

FIG. 15B shows the possible heteroduplex molecules formed in a one cycle regime. Note that ligation occurs only in the trimolecular heteroduplex molecule, generating the SEB products shown in FIG. 15C.

FIG. 15C shows the SEB products generated when starting with three DNA precursors using the HTLA system described herein.

FIG. 16 shows a strategy to generate an admixture of SEBs and blunt-ended products using the CHTLA system of the present invention with three DNA precursors and multiple cycles. Note uppercase lettering (A, B, C) denotes ‘top’ strands of DNA precursors and lowercase lettering (a, b, c) denotes ‘bottom’ strands.

FIG. 17 show a strategy to generate SEB with four DNA precursors using the HTLA system of the present invention and results of one cycle. Note uppercase lettering (A, B, C) denotes ‘top’ strands of DNA precursors and lowercase lettering (a, b, c) denotes ‘bottom’ strands.

FIG. 18 shows a strategy to generate an admixture of SEBs and blunt-ended products using the CHTLA system of the present invention with four DNA precursors and multiple cycles. Note uppercase lettering (A, B, C) denotes ‘top’ strands of DNA precursors and lowercase lettering (a, b, c) denotes ‘bottom’ strands.

FIG. 19 shows CHTLA performed with four single-stranded 5′-phosphorylated DNA precursor oligonucleotides. Note that the heating/melting step denatures any base pairing due to self-complementarity within each individual oligonucleotide. Note that the product formed is exclusively a single SEB, e.g., no blunt-ended products form when the DNA precursors are all oligonucleotides.

FIG. 20 shows the strategy to join 7 single stranded DNA oligonucleotides (I through VII) to form a 248 base-long SEB wherein 240 base pairs form, and 4-nucleotide sticky ends are present (colored lines), and the products of 5-cycle and 10-cycle CHTLA reactions with 7 DNA oligonucleotides (left gel image, 5× & 10×), and the results of colony PCR (16 colonies) upon ligation of the 5× HTLA products into a plasmid vector with compatible 4-base sticky ends and transformation into E. coli. Note that the results of the colony PCR demonstrate that 15/16 of the resultant clones carried the expected sized 240 base pair insert, the exception being shown in Lane 12. Sanger DNA sequencing of the DNA from colonies represent in Lanes 11and 12 showed that the colony represented in Lane 11 had the expected 240 bp DNA sequence, and the colony represented in Lane 12 had a tandem repeat of the 240-bp DNA sequence.

### DETAILED DESCRIPTION OF THE INVENTION

The present invention provides a Heteroduplex Thermostable Ligation Assembly (HTLA) method to generate sticky ends of user-defined length and sequence that is restriction endonuclease and DNA exonuclease activity-independent. The formed heteroduplex DNAs are joined by one or more ligation cycles using a thermostable DNA ligase, and wherein the formed heteroduplex DNAs generate, entirely in vitro, sticky-end blocks, an admixture of sticky end blocks and blunt-ended products, or covalently closed circular DNAs (cccDNAs), in which all of the foregoing may or may not contain variable regions, using the HTLA or Cyclic Heteroduplex Thermostable Ligation Assembly (CHTLA) method of the present invention.

### Definitions

As used herein, the term ‘heteroduplex’ DNA molecule refers to a mostly a double-stranded molecule wherein the ‘top’ strand originates from one double-stranded or single stranded precursor DNA molecule, and the ‘bottom’ strand originates from a different double-stranded or single-stranded DNA molecule, having been joined by Watson-Crick base pairing in the process known as annealing of, or hybridization of, complementary DNA strands.

As used herein, the term “ligase” and “ligation agent” are used interchangeably and refer to any number of enzymatic or non-enzymatic reagents capable of joining a linker probe to a target polynucleotide. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase. The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits.

As used herein, the term “overlapping sequence”, refers to a sequence that is complementary in two polynucleotides and where the overlapping sequence is single-stranded (ss), on one polynucleotide it can be hybridized to another overlapping complementary ss DNA region on another polynucleotide.

As used herein the term “overhang” refers to the single stranded region of double-stranded (ds) DNA at the end thereof and is either of type 5′ or 3′ due to the inherent directionality of DNA. The overhangs are generally generated in various lengths by treating dsDNA with restriction enzymes or exonucleases and/or by the addition of appropriate dNTPs (dATP, dTTP, dCTP, dGTP) through the action of an enzyme, i.e., terminal deoxynucleotidyl transferase.

As used herein the term double stranded DNA (dsDNA) refers to oligonucleotides or polynucleotides having 3′ overhang, 5′ overhang or blunt ends and composed of two single strands all or part of which are complementary to each other, and thus dsDNA may contain a single stranded region at the ends and may be synthetic or natural origin derived from cells or tissues. In one embodiment, dsDNA is a product of PCR (Polymerase Chain Reaction) or fragments generated from genomic DNA or plasmids or vectors by a physical or enzyme treatment thereof.

As used herein, the term “buffering agent”, refers to an agent that allows a solution to resist changes in pH when acid or alkali is added to the solution. Examples of suitable non-naturally occurring buffering agents that may be used in the compositions, kits, and methods of the present invention include, for example, Tris-HCL, MgCl₂, KCl, NAD, DTT, Triton X-100Tris, HEPES, TAPS, tricine. Other buffers include without limitation, phosphate, citrate, ammonium, acetate, carbonate, tris(hydroxymethyl)aminomethane (TRIS), 3-(N-morpholino) propanesulfonic acid (MOPS), 3-morpholino-2-hydroxypropanesulfonic acid (MOPSO), 2-(N-morpholino)ethanesulfonic acid (MES), N-(2-Acetamido)-iminodiacetic acid (ADA), piperazine-N,N′-bis(2-ethanesulfonic acid) (PIPES), N-(2-Acetamido)-2-aminoethanesulfonic acid (ACES), cholamine chloride, N,N-Bis(2-hydroxyethyl)-2-aminoethanesulfonic acid (BES), 2-[[1,3-dihydroxy-2-(hydroxymethyl)propan-2-yl]amino]ethanesulfonic acid (TES), 4-(2-hydroxyethyl)-1-piperazine ethanesulfonic acid (HEPES), acetamidoglycine, tricine (N-(2-Hydroxy-1,1-bis(hydroxymethyl)ethyl)glycine), glycinamide, and bicine (2-(Bis(2-hydroxyethyl)amino)acetic acid) buffers.

As used herein, the terms DNA or RNA is defined as a “polynucleotide” and may encompass primers, oligonucleotides, nucleic acid strands, etc. The DNA or RNA may be single stranded or double stranded or an admixture thereof. Such DNA or RNA polynucleotides may be synthetic, for example, synthesized in a DNA synthesizer, or naturally occurring, for example, extracted from a natural source, or derived from cloned or amplified material. Polynucleotides referred to herein may contain modified bases. Additionally, the DNA or RNA sequences may comprise one or more random or variable nucleotides. The use of randomized (ATCGNNNNATGC) may also include sequence-restricted regions, wherein sequence restricted means limiting the variation at one position to 2 or 3 nucleotide choices (i.e. A or C; A, G, or C etc.) rather than all 4 (ATGC). Typically, a polynucleotide contains a 5′ phosphate at one terminus (“5′ terminus”) and a 3′ hydroxyl group at the other terminus (“3′ terminus) of the chain.

The nucleic acids utilized herein can be any nucleic acid, for example, human nucleic acids, bacterial nucleic acids, or viral nucleic acids. The nucleic acid sample can be, for example, a nucleic acid sample from one or more cells, tissues, or bodily fluids such as blood, urine, semen, lymphatic fluid, cerebrospinal fluid, or amniotic fluid, or other biological samples, such as tissue culture cells, buccal swabs, mouthwashes, stool, tissues slices, biopsy aspiration, and archeological samples such as bone or mummified tissue. Nucleic acids can be, for example, DNA, RNA, or the DNA product of RNA subjected to reverse transcription. Nucleic acids can be derived from any source including, but not limited to, eukaryotes, plants, animals, vertebrates, fish, mammals, humans, non-humans, bacteria, microbes, viruses, biological sources, serum, plasma, blood, urine, semen, lymphatic fluid, cerebrospinal fluid, amniotic fluid, biopsies, needle aspiration biopsies, cancers, tumors, tissues, cells, cell lysates, crude cell lysates, tissue lysates, tissue culture cells, buccal swabs, mouthwashes, stool, mummified tissue, forensic sources, autopsies, archeological sources, infections, nosocomial infections, production sources, drug preparations, biological molecule productions, protein preparations, lipid preparations, carbohydrate preparations, inanimate objects, air, soil, sap, metal, fossils, excavated materials, and/or other terrestrial or extra-terrestrial materials and sources.

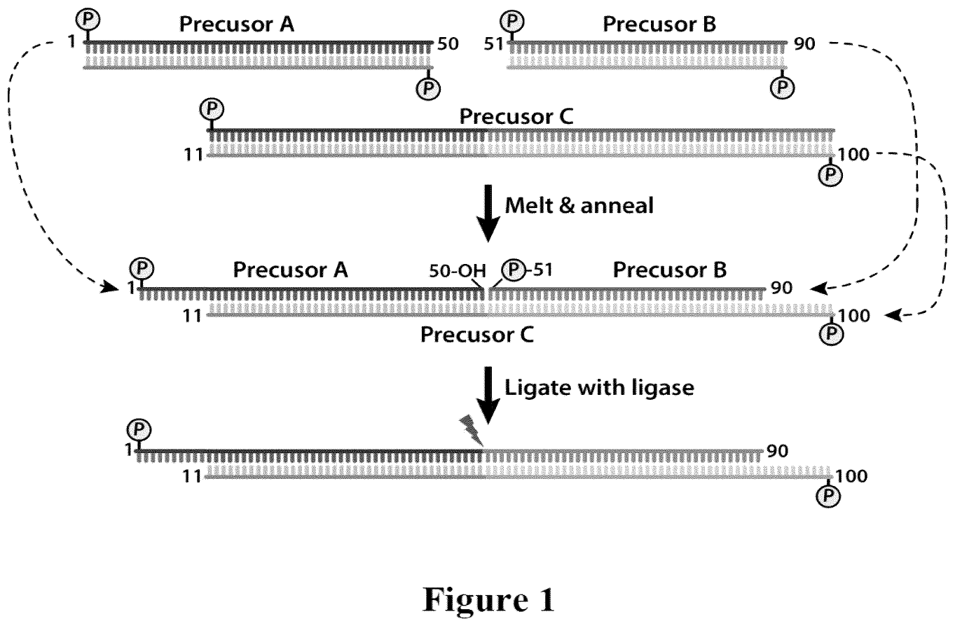

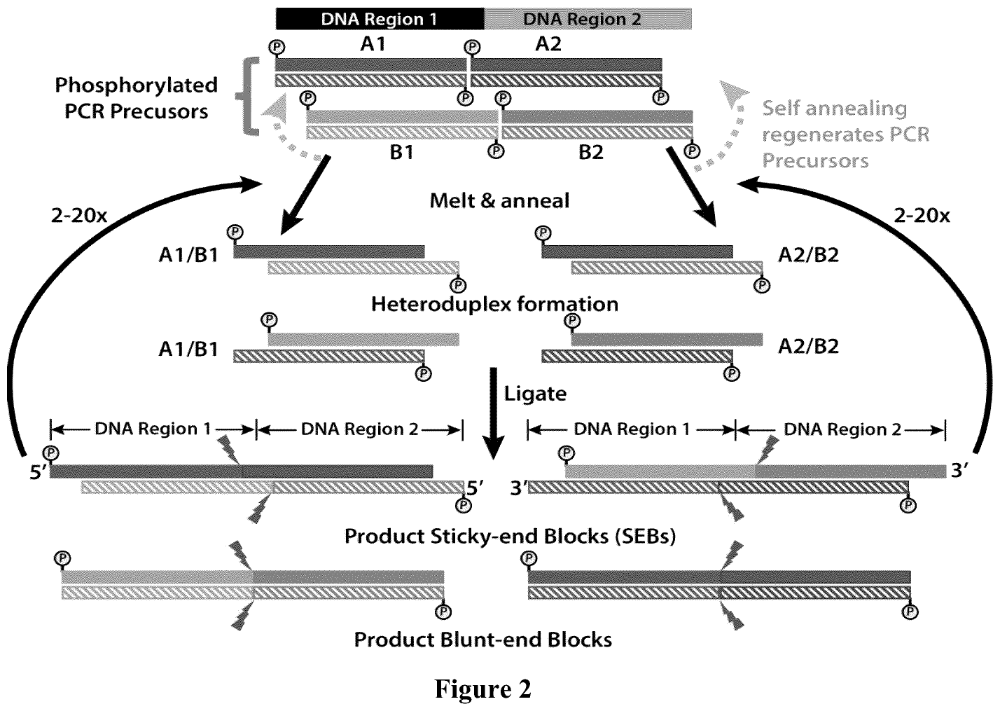

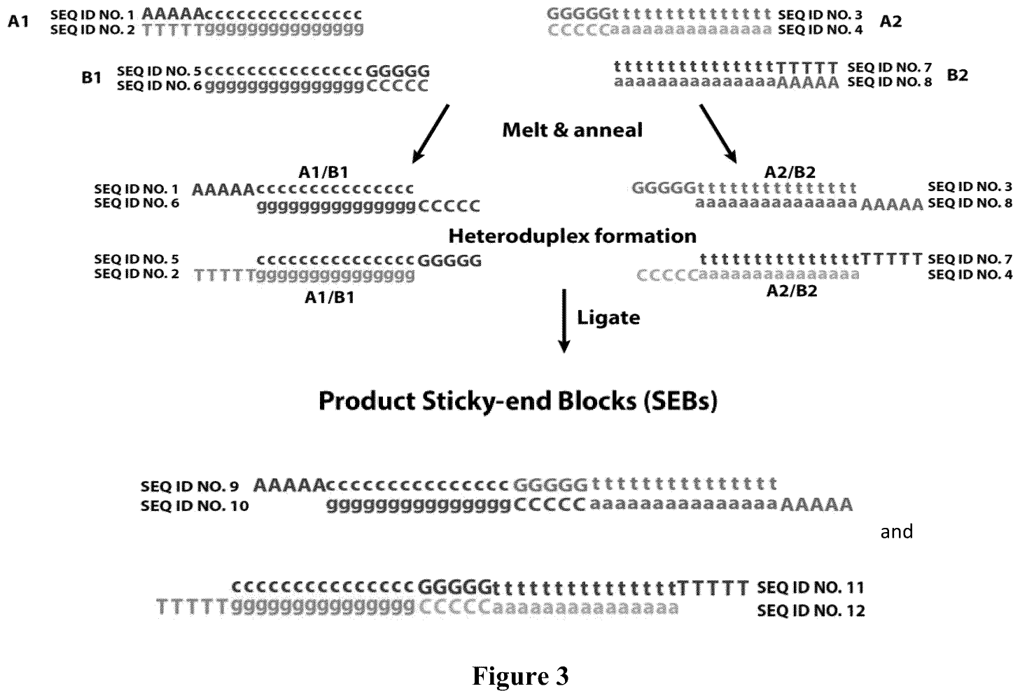

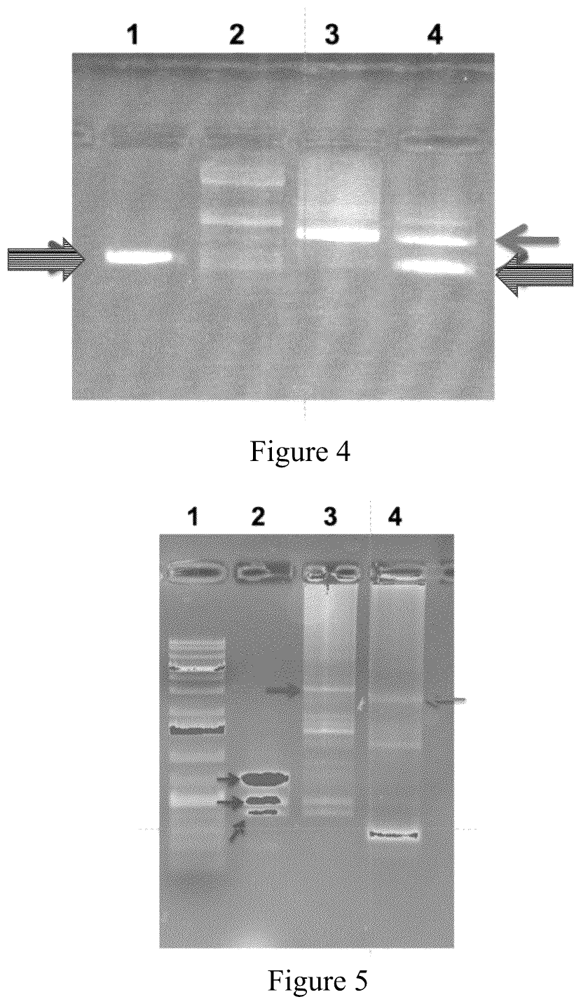

The invention disclosed herein uses precursor DNA fragments that undergo one or more cycles of thermal denaturation, hybridization, and ligation using a thermostable DNA ligase in a thermal cycler in a specific buffer. Recognizing the urgent need for more efficient, high-fidelity technologies for large DNA assembly, Heteroduplex Thermostable Ligase Assembly (HTLA) was developed by the present inventors. HTLA is a straightforward assembly platform that generates ligation-ready single-stranded overhangs of user-defined length to create sticky-end blocks (SEBs) for assembly into higher order linear or circular structures. The HTLA/CHLTA process generates a product termed a Sticky-End Block (SEB) that consists of a double-stranded DNA molecule with single-stranded ends as shown in FIG. 1 and FIG. 2.

Notably the precursors of FIG. 1 and all precursors described herein include the creation of an SEB by HTLA or CHTLA wherein the precursor DNAs are specifically designed. For example, consider the simple case for the design of three double stranded precursors (A, B, & C) needed to create a 100 base-pair SEB wherein 80 base pairs are double stranded, and the 5′ end has a 10-base sticky end, and the 3′ end has a 10-base sticky end. Precursor A could be a double-stranded molecule comprised of nucleotides 1-50. Precursor B could be a double stranded molecule comprised of nucleotides 51-90. Precursor C could be a double stranded molecule comprised of nucleotides 11-100. Thus, Precursors A and C are identical in DNA sequence from nucleotides 11-50, and Precursors B and C are identical in DNA sequence from nucleotides 11-90. Upon melting and reannealing, the so-called top strands of Precursors A and B can anneal to the so-called bottom strand of Precursor C due to the complementarity of the nucleotide sequences from nucleotides 11-50 in Precursor A and from 51-90 in Precursor B, yielding an 80 base-pair double-stranded region. Since nucleotides 1-10 of Precursor A do not have complementary bases in Precursor C, they remain single stranded. Likewise, nucleotides 91-100 in Precursor C do not have complementary bases in Precursor B, so they remain single stranded. The so-called nick in the phosphodiester backbone between nucleotides 50 and 51 on the top strand of the heteroduplex can be sealed by a DNA ligase. Thus, a 100 base-pair SEB is formed wherein an 80 nucleotide-long region is double stranded (from nucleotides 11-90), and the 5′ and 3′ ends bear a 10 nucleotide-long sticky end. Note that the converse heteroduplex formation also occurs, that is, the bottom strands of Precursors A and B can form complementary base pairs with the top strand of Precursor C and be ligated to form an SEB. It is axiomatic that, in addition to the formation of SEB products, the three Precursors A, B, and C can also reform by the re-annealing of their complementary strands.

A schematic of SEB generation is shown in FIG. 2 wherein 5′-phosphorylated overlapping (and offset) synthetic DNAs, (i.e., PCR products, de novo synthesized DNAs (single-stranded or double-stranded), standard restriction enzyme-generated fragments or a combination thereof were denatured in a thermocycler and allowed to anneal in the presence of a thermostable ligase. Upon annealing, in addition to re-annealed input DNAs (Precursors), heteroduplex DNAs with 5′ and 3′ overhangs are formed. For example, to make an SEB product joining, from left to right, Region 1 to Region 2, four precursor PCR fragments are generated. The left end of Precursor A1 coincides with the left end of Region 1, and its right end is short of the right end of Region 1. The left end of Precursor B1 is internally offset from the left end of Region 1. The extent of this offset defines the length of the left sticky end (i.e., 4 to >100 bp). The right end of Precursor B1 extends beyond the right end of Region 1 and into Region 2. The extent of intrusion into Region 2 defines the length of overlap between Precursor B1, and Precursor A2, one of a second pair of precursor PCR products (A2 and B2) representing Region 2. The left end of Precursor A2 resides in Region 1 and overlaps with Precursor B1. The right end of Precursor A2 is short of the right end of Region 2. The left end of Precursor B2 is internally recessed from the left end of Precursor A2, and its right end coincides with the right end of Region 2. The extent of offset on the right ends of Precursors A2 and B2 defines the length of the right sticky end (i.e., 4 to >100 bp).

When the four precursor PCR fragments are melted and reannealed, there are eight possible outcomes. The single strands of the four Precursor PCR fragments can come back together, regenerating the input Precursors (broken arrow, FIG. 2). In addition, four heteroduplexes can form: two A1/B1 heteroduplexes (i.e., top strand of A1 annealed to bottom strand of B1, and vice versa) and two A2/B2 heteroduplexes (i.e., top strand of A2 annealed to bottom strand of B2, and vice versa). Note that the offset between the ends of A1 and B1 result in one heteroduplex with 5′ single-stranded overhangs, and one heteroduplex with 3′ single-stranded overhangs. The same holds true for A2/B2 heteroduplexes. Precursors are designed such that the overhangs on the right end of A1/B1 heteroduplexes are perfectly complementary to overhangs on the left end of A2/B2 heteroduplexes. This complementarity allows A1/B1 heteroduplexes to be ligated to A2/B2 heteroduplexes, generating the SEB product, a double-stranded DNA molecule with non-complementary sticky ends, now consisting of Region 1 and Region 2. Note that the ligation of heteroduplexes reduces the pool of Precursor PCR fragments. When a thermostable ligase is used, then after the ligation reaction, the temperature can be raised to 98 degrees or higher to melt both Precursor PCR fragments and Product SEBs. Upon cooling, ligation of A1/B1 to A2/B2 heteroduplexes occurs again, generating more Product SEBs and further depleting the pool of Precursor PCR fragments. After a number of cycles, a substantial portion of the Precursor PCR fragments are converted to a single SEB product consisting of DNA Region 1 joined to DNA Region 2, largely double-stranded, but with non-complementary single stranded ends. Note that this description illustrates the formation of a relatively simple SEB, from four PCR Precursors joining DNA Region 1 to DNA Region 2. More complex SEBs can also be formed by HTLA from 5 to >100 Precursors.

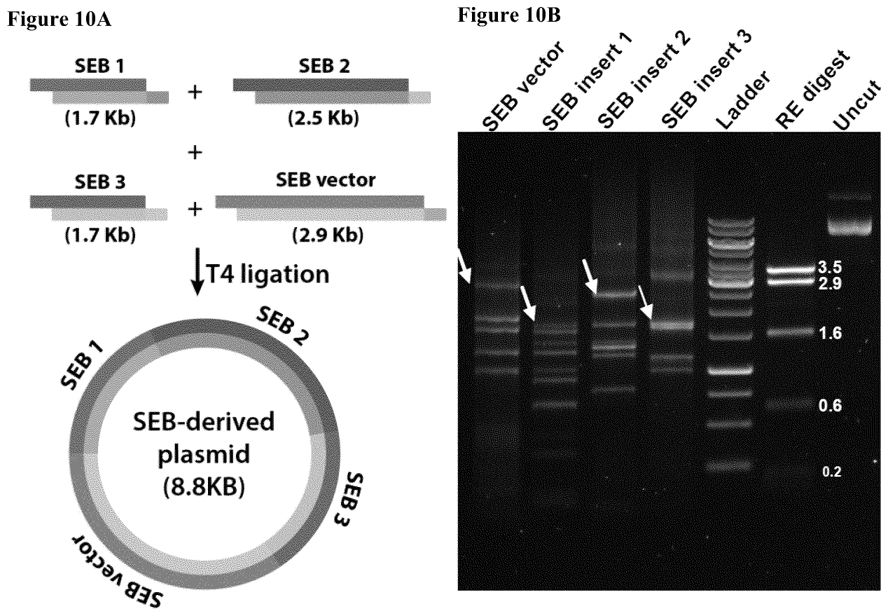

In one embodiment, to link DNA fragments in an ordered array, multiple SEBs are generated in separate reactions and purified (i.e., by gel purification), and their final single stranded overhangs are designed to be complementary, that is, the right sticky end of SEB 1 is perfectly homologous to the left sticky end of SEB 2, and so on. The multiple SEBs so designed can then be ligated by a conventional DNA ligase (i.e., T4 DNA ligase) in a non-cyclic manner to generate a larger DNA molecule consisting of multiple joined SEBs as shown in FIG. 10. If a vector (plasmid, cosmid, BAC, YAC, etc.) is included as an SEB, and its sticky ends are complementary to the first and last SEBs in the ordered array, then a complete closed circular vector that can be propagated in bacteria or yeast results as shown in FIG. 10.

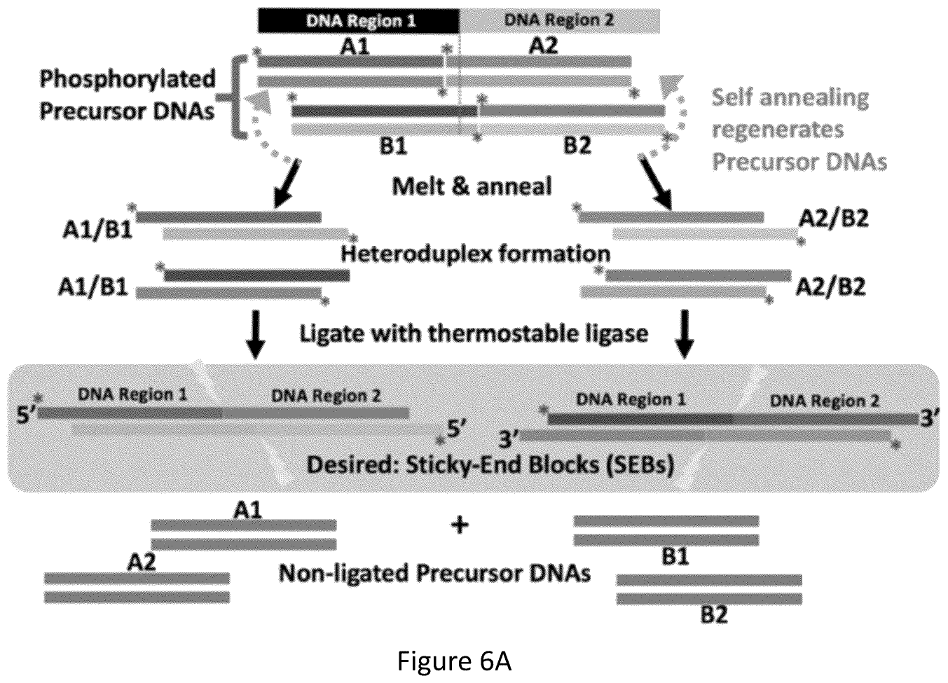

In another embodiment, multiple SEBs are created in a single reaction in a multiplexed manner, such that their overhangs are homologous, and are designed such that the SEBs can be joined in an ordered manner by HTLA/CHTLA alone as they are being generated from Precursor DNA fragments (i.e they are not individually purified then ligated). SEBs joining DNA Region 1 to DNA Region 2, and DNA Region 2 to DNA Region 3, and DNA Region 3 to DNA Region 4, etcetera, joining even tens-to-thousands of DNA regions in a single reaction vessel, can be generated by HTLA/CHTLA using DNA precursors. In this case, if a vector (plasmid, cosmid, BAC, YAC, etc.) is included as an SEB, and its sticky ends are complementary to the first and last SEBs in the ordered array, then a complete closed circular vector that can be propagated in bacteria or yeast results. An example is shown in FIG. 5 that demonstrates CHTLA using 10 DNA precursors to join five DNA Regions in a single reaction vessel.

In another embodiment, a vector sequence is not included among the Precursor sequences, and the first and last DNA elements of the ordered linear assembly bear complementary sticky ends. In this case, a cccDNA, aka a DNA minicircle, will be formed by the HTLA/CHTLA process.

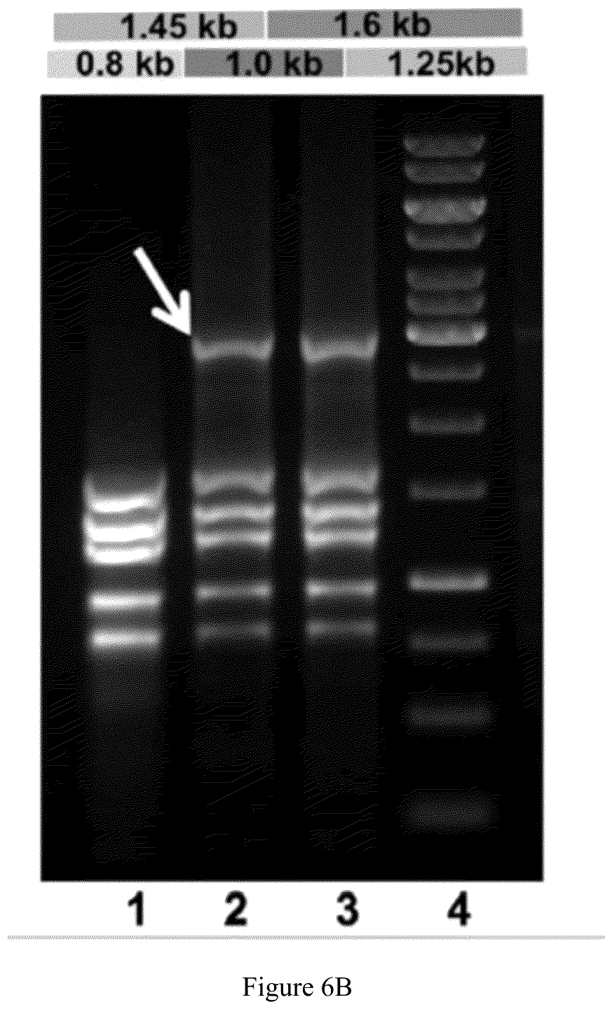

FIGS. 6A and B shows the HTLA of the present invention. FIG. 6A shows four (or more) offset and partially overlapping phosphorylated precursors from two (or more) DNA regions to be assembled are melted and heteroduplexes are ligated during annealing in a temperature gradient. Shaded box shows the desired sticky-end blocks (SEBs). FIG. 6B shows the gel image from a 5-precursor HTLA to generate the 3.0 kb ORF of the human gene SAP130. Lane 1, 1.6, 1.45, 1.25, 1.0, & 0.8 kb precursors; lane 2, HTLA reaction products using Ampligase thermostable ligase; Lane 3, HTLA reaction products using HiFi Taq thermostable ligase; Lane 4 DNA marker. Arrow, SEB product. * indicates 5′-PO₄.

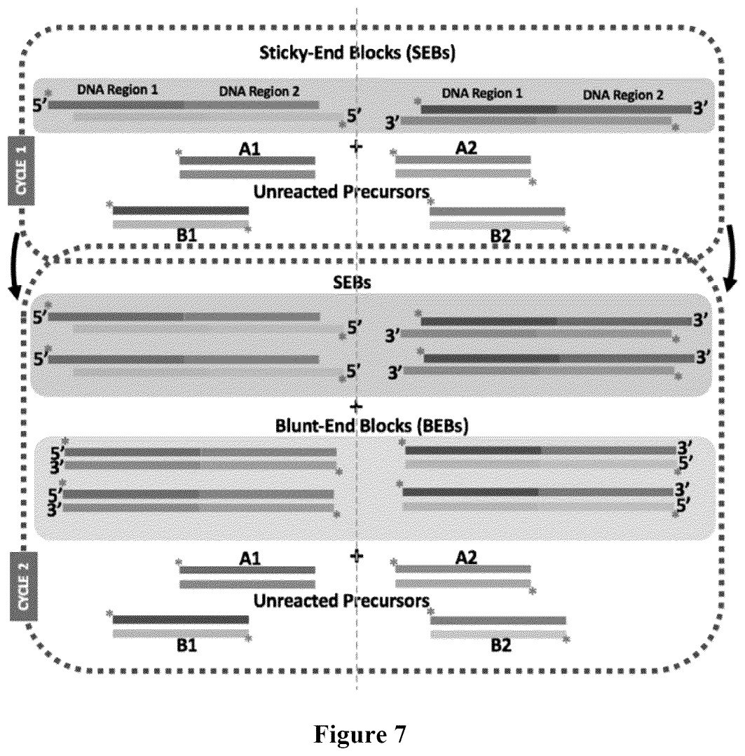

FIG. 7 shows the steps for Cyclic Heteroduplex thermostable ligase assembly (CHTLA) of the present invention. SEB products at the end of cycle 1 are melted in cycle 2. A mix of SEBs and blunt-end blocks (BEBs) form upon annealing in cycle 2 and subsequent cycles. * indicates 5′-PO₄.

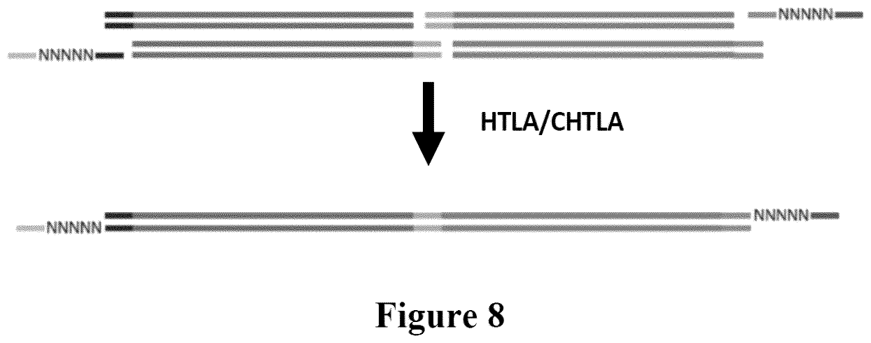

In another embodiment, oligonucleotides can be added to the HTLA/CHTLA and be incorporated into SEBs. The ability to include oligos with randomized regions is very important. It allows for the generation of pools of large DNAs that have identical sequences except for precise locations that are randomly mutated or limited in sequence variation (i.e., only a purine at a given position or only a pyrimidine at a given position). One application of this is to create bacteriophage libraries for phage with large genomes that have mutated receptor binding motifs which will be discussed hereinbelow. Notably, such libraries can be screened for commercially valuable phage for many applications. Using this strategy as shown in FIG. 8, a 1.6 kb SEB was generated, cloned, and sequenced, and 5 out of 5 had the mutation on both ends.

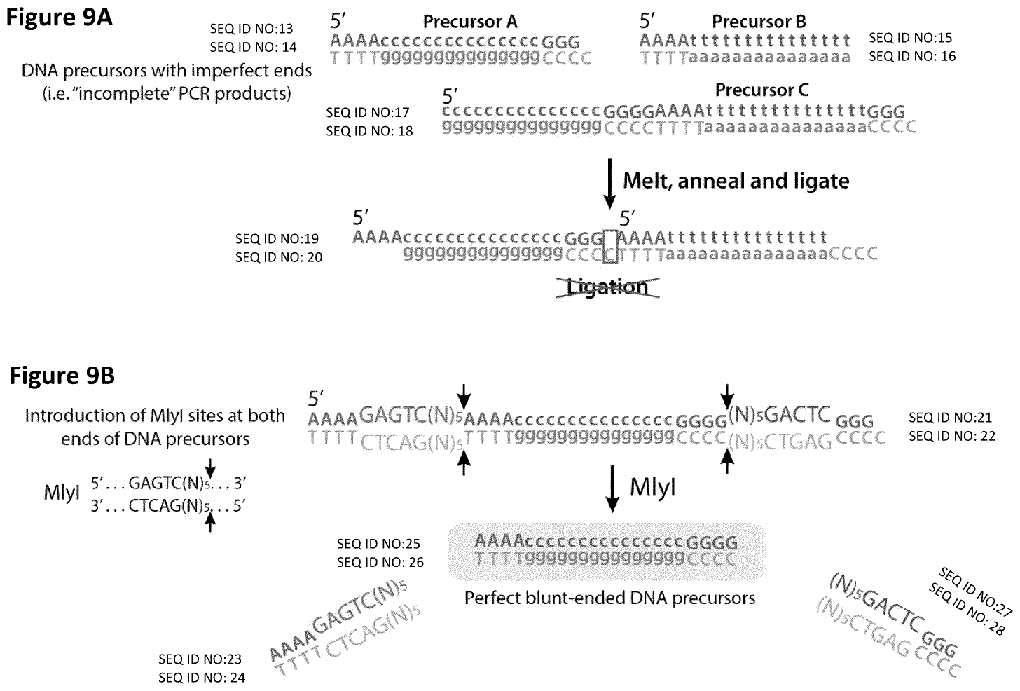

In another embodiment, PCR precursors are ‘polished’ to create DNA ends of a defined nature that may be perfect blunt ends as shown in FIG. 9, or sticky ends of a defined sequence. PCR reactions generate a substantial portion of products with incomplete 3′ ends that are thus undefined. Such ‘short’ or incomplete PCR products are not useful to serve as precursors in HLTA or CHTLA reactions. For example, to create blunt-ended products, a Type IIS restriction enzyme (i.e., MlyI) recognition sequence is included in the PCR primers used to generate PCR-generated DNA precursors for an HLTA or CHTLA reaction. After PCR, the product is cut with the Type IIS enzyme to create a pool of perfectly blunt ended precursors for the HLTA or CHTLA assembly reaction. In another example, to create a DNA precursor with defined sticky ends, the Type IIS enzymes BsaI or PaqCI or BsmBI or BspQI would be used to digest the PCR-generated DNA precursors prior to use in an HTLA or CHTLA reaction.

To demonstrate that multiple SEBs can be generated, purified, and ligated to create an ordered DNA assembly in a plasmid vector, four SEBs were generated that when correctly assembled, would yield an 8.8 kb circular plasmid (FIG. 10). HTLA products are shown in FIG. 10B. The strategy used for a four-SEB ligation includes a 2.9 kb vector SEB that was generated in a one-cycle HTLA reaction from 4 PCR precursors (1.2 kb, 1.7 kb, 1.9 kb and 1.0 kb). Three SEB inserts were built in 10-cycle CHTLAs (95° C. for 1 minute, 60° C. for 5 minutes×10) as follows: SEB 1 (1.7 kb final), from 0.35 kb, 1.35 kb, 0.95 kb, and 0.75 kb precursors; SEB 2 (2.5 kb final) from 1.6 kb, 0.9 kb, 1.7 kb, and 0.8 kb precursors; SEB 3 (1.7 kb final) from 0.6 kb, 0.6 kb, 1.1 kb and 1.1 kb precursors. Reaction mixture DNA was purified and ligated at room temperature with T4 DNA ligase. SEB sticky ends=6 nucleotides. T4 ligation products were transformed into DH5α. Precursor offset ensures perfect complementarity between heteroduplexes, and their juxtaposed ends are readily ligated to generate an SEB. Although CHTLA reactions generate multiple dead-end intermediate products, correct-size SEB yield is sufficiently high to generate a crisp band that can be gel-purified (white arrows, FIG. 10B). To date, this straightforward strategy to generate dsDNAs with complementary overhangs to use as building blocks for larger assemblies has never been conceived.

Single- and multiple-cycle HTLA products are shown in FIG. 10B. Correctly ordered assemblies were identified by colony PCR and confirmed by restriction enzyme digestion (FIG. 10B). Correct clones identified by colony PCR were confirmed in FIG. 10B and these data demonstrate that CHTLA-generated SEBs can be efficiently joined into higher order assemblies. Note that, correct-size SEB yield, comprised of a mixture of sticky-ended and blunt-ended products is sufficiently high to generate a crisp band that can be purified by conventional agarose gel purification or other means (FIG. 10B). These data demonstrate that HTLA- and CHTLA-generated SEBs can be efficiently joined into higher order assemblies.

In another embodiment, a continuous chain of SEBs is created in a single reaction in a multiplexed manner, such that their overhangs are complementary, and are designed such that the SEBs can be joined in an ordered manner by cyclic ligation alone as they are being generated from precursor fragments (i.e they are not individually purified then ligated by, for example, T4 ligase). Despite the fact that many methods are available to generate randomized DNA libraries, a robust method to generate random mutations at multiple regions in a large, circular DNA (>50 kb) in a seamless manor is currently unavailable.

The method of the present invention will be useful to generate not only phage libraries, but will also be a powerful method to generate other viral libraries where mutations in several dispersed locations may be beneficial, for example, in adeno-associated virus libraries that are being screened to find tissue-tropic isolates for gene therapy applications. These goals were achieved in the present invention by incorporating single-stranded oligos with degenerate regions into SEBs in CHTLA reactions.

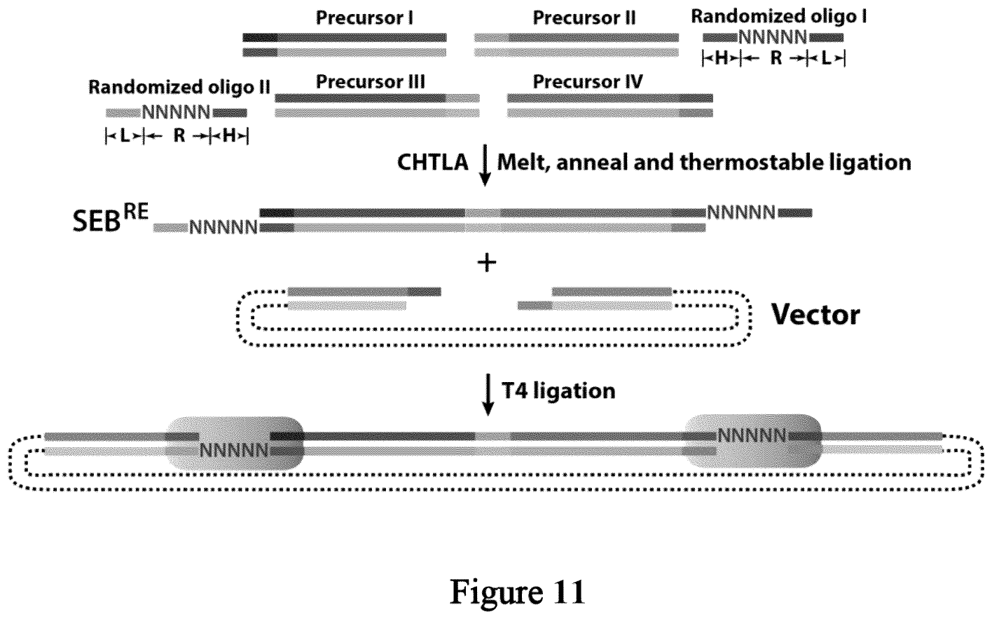

In yet another embodiment, a single stranded oligonucleotide can be one of the components in the HTLA or CHTLA reaction. Furthermore, the oligonucleotide can contain a variable region such that the reaction generates an SEB containing a variable DNA sequence of known length and potential sequence, for example, to generate a so-called library of SEBs that can be incorporated into a larger DNA molecule such that the final large DNA contains one or more regions of variable DNA sequence. In one example, oligonucleotides were used to generate variable or randomized regions near the end of the SEB, which we term stick-end block with randomized end, or SEB^{RE}. To demonstrate feasibility of SEB^{RE} generation, as shown in FIG. 11, two 40-mer oligos were incorporated into a 1.7 kb SEB (one on each end). Region H=29 bases, R=5 bases, L=6 bases by 10-cycle CHTLA. Notably the length of the region L length can be from about 6 to 50 and the region R length can be varied from 6 to 100. The SEB^{RI} was ligated into a 2.9 kb vector SEB with T4 DNA ligase. Positive clones were identified by colony PCR (20/20), & successful oligos incorporation (both) was confirmed by DNA sequencing (5/5).

Specifically, a CHTLA reaction was performed to generate a 1.7 kb SEB and added two ‘model’ oligos. The oligos featured end homology to PCR precursors, FIG. 11, region H,) end complementarity to a ligation partner SEB (FIG. 11, region L) and five central, non-complementary bases (FIG. 11, region R). Ligation of the oligo-modified SEB with model ‘random ends’ (SEB^{RE}, note that the randomized region is just internal to the sticky-end sequence required to direct ordered ligation) to an SEB-generated vector yielded ˜1000 colonies. 20/20 were correct by colony PCR, and sequencing revealed that both oligos were incorporated in 5/5 clones. These data demonstrated the feasibility of this approach. In this case also, if a vector (plasmid, cosmid, BAC, YAC, etc.) is included as an SEB is generated by linking several SEBs, and its sticky ends are complementary to the first and last SEBs in the ordered array, then a complete closed circular vector that can be propagated in bacteria or yeast results.

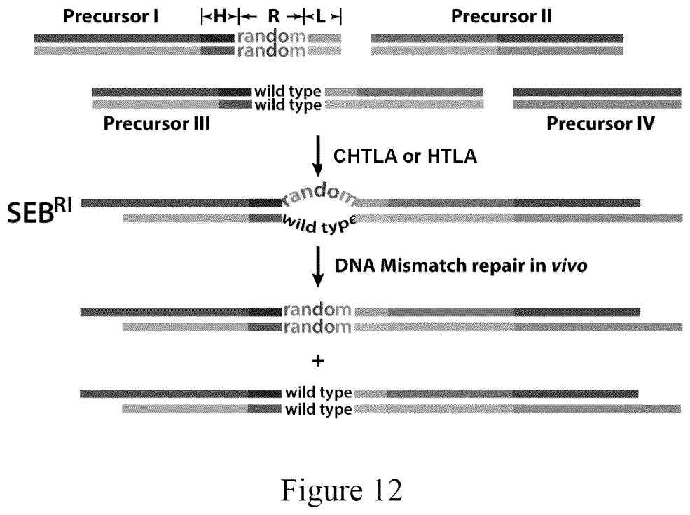

While developing CHTLA, it was determined that degenerate oligos can be used to generate PCR precursors that give rise to SEBs with internally-located randomized regions SEBs (SEB^{RIs}) are well tolerated, and SEB^{RIs} are as efficiently ligated as ‘regular’ SEBs (FIG. 12). This observation inspired a distinct technical strategy to introduce random mutations in SEBs for library diversification. Specifically, a precursor was generated with a randomized or variable region, for example, by PCR, and that precursor was used to generate a sticky end block with an internal randomized region, or SEB^{RI}. To test feasibility, as shown in FIG. 12, two internal mismatched regions were introduced into a 1.7 kb SEB. Region R=5 bases, region H=29 bases, and region L=6 bases. Mismatched region 1 was on the SEB leading strand at position 227-231; mismatched region 2 was on the lagging strand at position 790-794. The 1.7 kb SEB^{RI} was ligated to a 2.9 kb SEB vector (6-base sticky ends) with T4 DNA ligase. Correct clones were identified by colony PCR, and 20 colonies were chosen for DNA mini-prep and DNA sequencing. Interestingly, the mismatch at position 227-231 (leading strand) was present in 17/20. In contrast, the mismatch at position 790-794 (lagging strand) was present in 3/20, potentially due to strand bias during DNA repair in vivo. In other experiments, an attempt to bias repair toward the randomizing oligo by methylation of adenosines in the SEB^{RI} using deoxyadenosine methylase. Despite confirmation of successful complete methylation using methylation-sensitive restriction enzyme digestion, no bias was found, contrary to the existing dogma in the E. coli DNA repair field (data not shown).

Another embodiment addresses the inexorable rise of multiple antibiotic resistant bacteria has led to a vigorous renewal of interest in bacteriophage (phage) therapy for patient treatment. However, phages are a largely untapped resource with potential to transform not only infectious disease control, but also food preservation, plant pathogen control, biosensor development, biofilm control, and surface disinfection. Phage exclusively infect and lyse bacteria with extraordinary species and strain specificity. Due to the large size of many phage genomes (up to 500 kb) and unknown function of many open reading frames, ensuring the environmental or patient safety of new isolates is an onerous yet essential task. Phage genome engineering has also been severely limited by the lack of packaging systems for all but a few ‘benchmark’ phage (i.e., lambda, M13). A recent breakthrough in phage manipulation has the potential to revolutionize this paradigm: it is now possible to “reboot” the majority of phage in the common, highly transformable E. coli strain DH10B, opening the door to create libraries of ‘wild’ phage. To exploit this breakthrough, there is a pressing need to develop efficient means of generating creating high complexity libraries using fully characterized “chassis” phage that vary in receptor binding motifs that dictate exquisite bacterial strain specificity. The present invention addresses the need for creating high complexity libraries by the synthesis of large DNAs with variable regions to enable complex chassis phage library construction. Current synthetic large DNA assembly technologies are technically incapable of achieving this goal. All ordered DNA assembly requires complementary single-stranded cohesive (sticky) ends.

The present invention provides a method to generate sticky ends of user-defined length and sequence that is restriction endonuclease and DNA exonuclease activity-independent.

Heteroduplex DNAs are joined by multiple ligation cycles using a thermostable DNA ligase and the present invention provides a novel approach, termed Cyclic Heteroduplex Thermostable Ligation Assembly (CHTLA) to generate, entirely in vitro, large covalently closed circular DNAs (cccDNAs) with variable regions. Notably, CHTLA of the present invention can be used to rapidly generate large, high complexity phage genome libraries and transform them into a third-party host. Such libraries would be a high-value resource that can be screened for phage with commercial potential for any application where phages are needed to control bacterial growth.

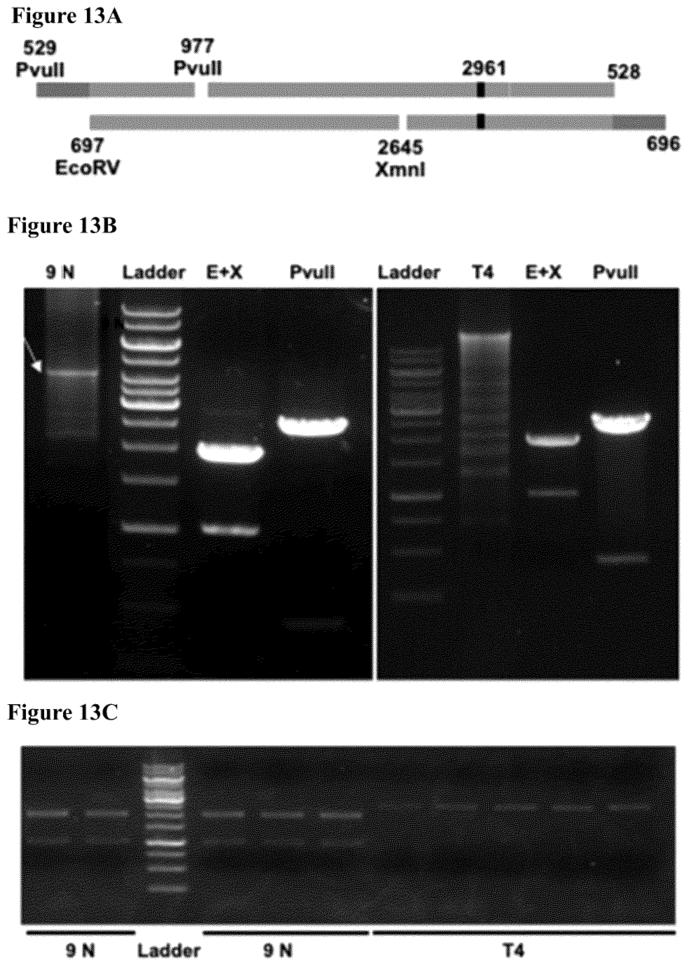

The present invention provides for a lambda genome that is ‘pre-circularize’ to increase transformation efficiency. As proof-of-principle to demonstrate that CHTLA can generate circular DNAs, four precursors were designed to build a 3 kb circular plasmid (FIG. 13B) using 9°N™ (9N) ligase in CHTLA. Transformation using relatively low competency cells (5×10⁷) yielded >10⁸ colonies, and 100% (5/5) colonies harbored correct assemblies (FIG. 13C). This efficiency exceeds, by several orders of magnitude, that which can be achieved using Gibson assembly or conventional cloning using T4 DNA ligase. To demonstrate this, conventional ligation of the same fragments with T4 ligase was performed and resulted in an array of unproductive ligation events (FIG. 13B) that yielded ˜10⁵ colonies, and 0/5 bore correct assemblies. These data demonstrate that CHTLA dramatically outperforms conventional cloning in the production of correctly assembled closed circular DNAs, with potential to yield >10¹⁰ colonies using super-competent (i.e. 10¹⁰/ug) cells.

FIG. 13 shows that the CHTLA of the present invention efficiently produces circular DNA. Specifically pBluescript II (SK-) (2.961 kb) was digested to generate all blunt-ended fragments using Pvu II alone, or Eco RV+Xmn I (E+X). Pvu II digestion produces two blunt DNAs, ˜2.51 kb and ˜0.45 kb, E+X digestion produces two blunt DNAs, ˜1.95 kb and 1.01 kb (FIG. 13, top panel). DNA was mixed, purified & ligated with 9°N™ (9N) thermo-stable ligase in a 10-cycle CHTLA reaction (FIG. 13, left middle panel). A product with apparent size of ˜4.5 kb is clearly visible on the gel (FIG. 13B, Lane 9N), which is the expected position of a closed circular 3 kb DNA. The purified ligation mixture (1 μl) was transformed into electrocompetent cells (competency=˜5×10⁷ colony/μg pUC19 DNA) yielding ˜5,000 colonies under antibiotic selection. In parallel, the same DNAs were ligated with T4 DNA ligase at room temperature (FIG. 13, right middle panel). A series of DNA fragments were formed (FIG. 13B, right middle panel, Lane T4), including products>10 kb. Transformation of 1 μl of the T4 ligation reaction yielded ˜80 colonies. 5 colonies were picked from both 9°N™ (9N) ligase plate and 5 from the T4 ligase and DNA was restriction digested with Eco RV+Xmn I (bottom panel). All five colonies from 9N plate gave the correct fragments. 0/5 T4 ligase plate colonies yielded the correct fragments.

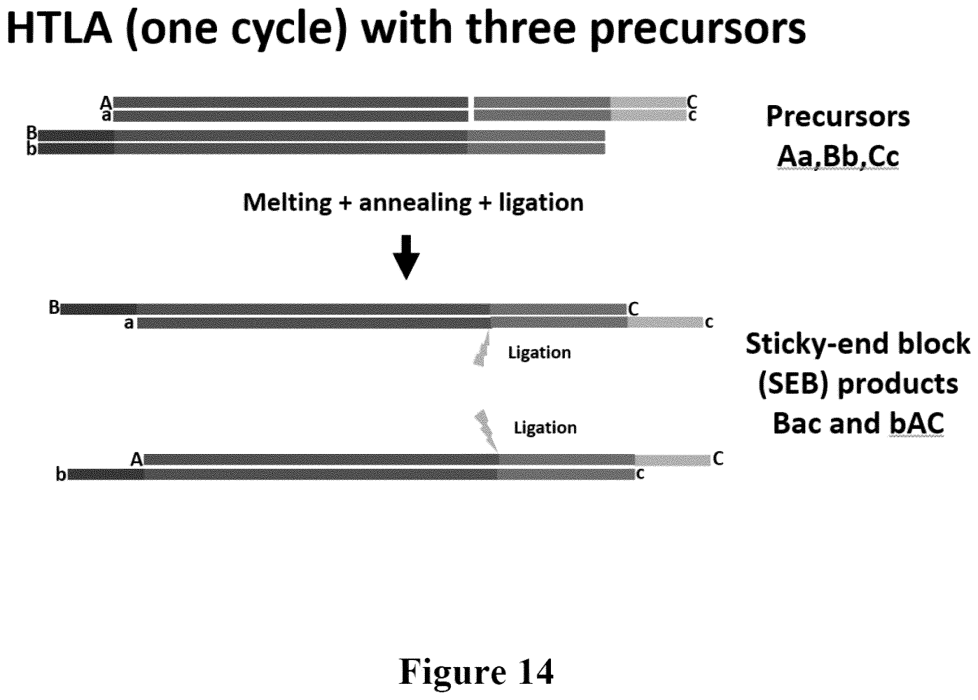

FIG. 14 show a strategy to generate SEB with three DNA precursors using the HTLA system of the present invention and results of one cycle. The three precursors of Aa, Bb and Cc are melted, annealed and ligation and form sticky-end blocks products Bac and bAC.

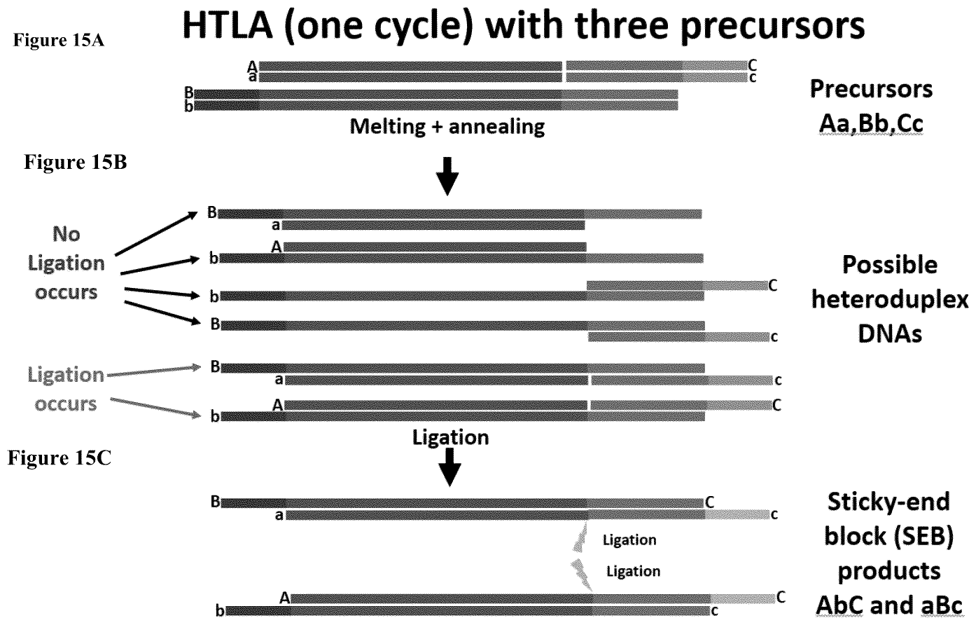

FIG. 15 shows a strategy to generate SEB with three DNA precursors using the HTLA system of the present invention and the possible HTLA DNAs formed in a one cycle regime. The three precursors of Aa, Bb and Cc are melted, annealed and ligation and several possible non-ligatable heteroduplex DNAs are formed. The sticky-end block products are AbC and aBc.

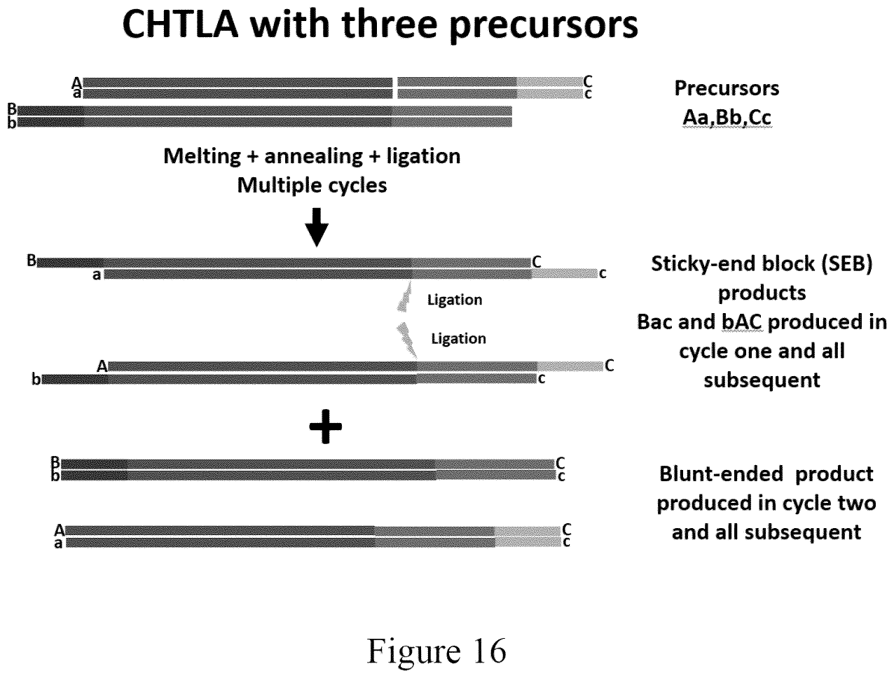

FIG. 16 shows a strategy to generate an admixture of SEBs and blunt-ended products using the CHTLA system of the present invention with three DNA precursors and multiple cycles. The three precursors of Aa, Bb and Cc are melted, annealed and ligation and the sticky-end Bac and bAC are produced and addition cycles of the melting, annealing, and ligation forms an admixture of the desired SEBs and blunt-ended products.

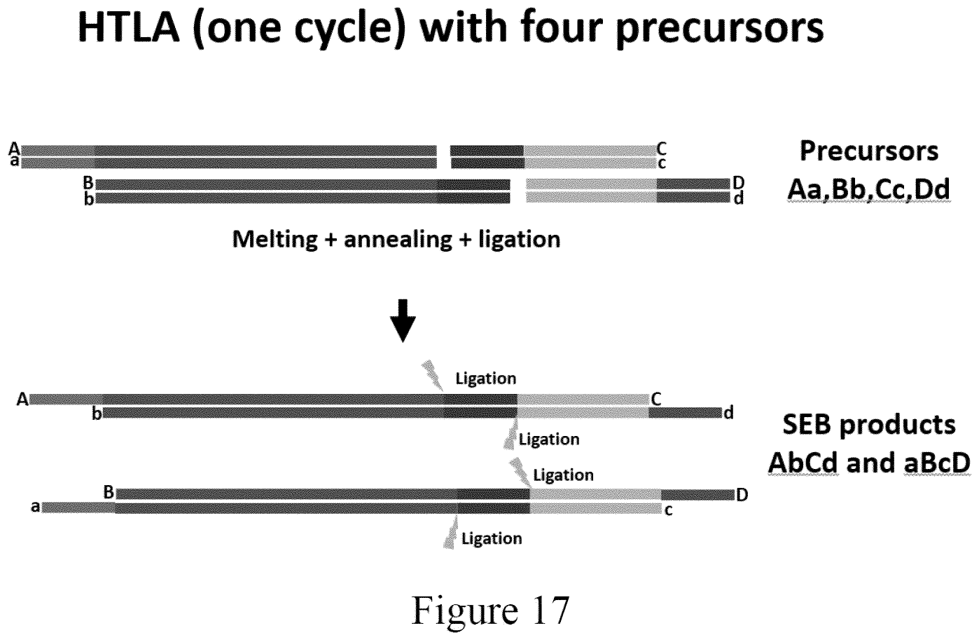

FIG. 17 show a strategy to generate SEB with four DNA precursors using the HTLA system of the present invention and results of one cycle. The four precursors of Aa, Bb, Cc and Dd are melted, annealed and ligation and several possible heteroduplex DNAs are formed. The sticky-end block products are AbCd and aBcD.

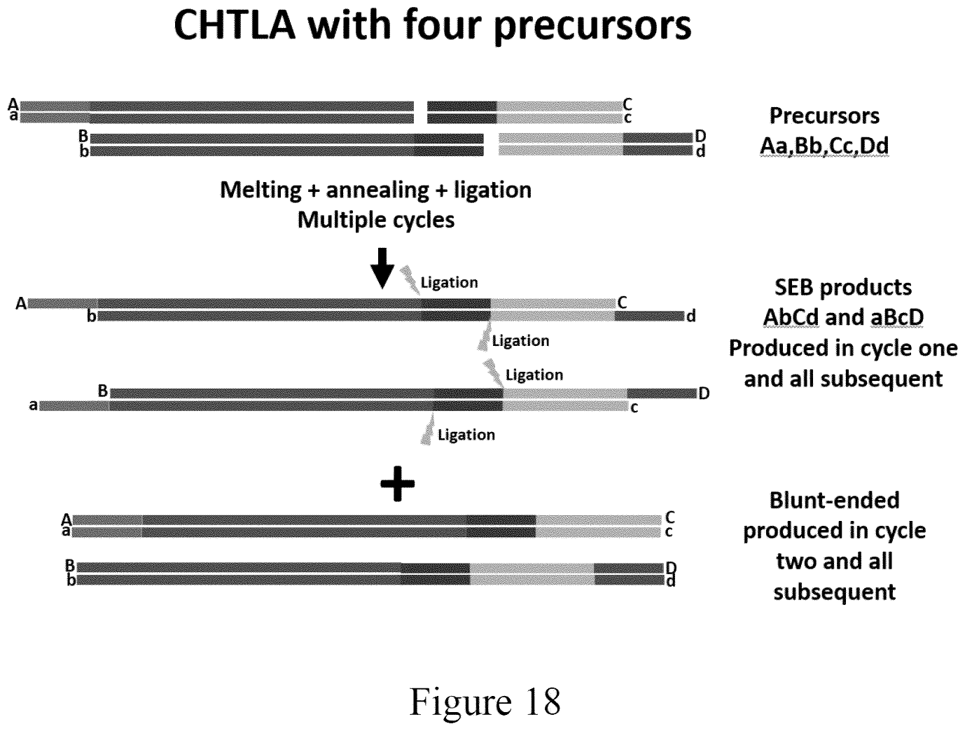

FIG. 18 shows a strategy to an admixture of SEBs and blunt-ended products using the CHTLA system of the present invention with four DNA precursors and multiple cycles. The four precursors of Aa, Bb, Cc and Dd are melted, annealed and ligation and the sticky-end AbCd and aBcD are produced and addition cycles of the melting, annealing, and ligation forms blunt-ended products.

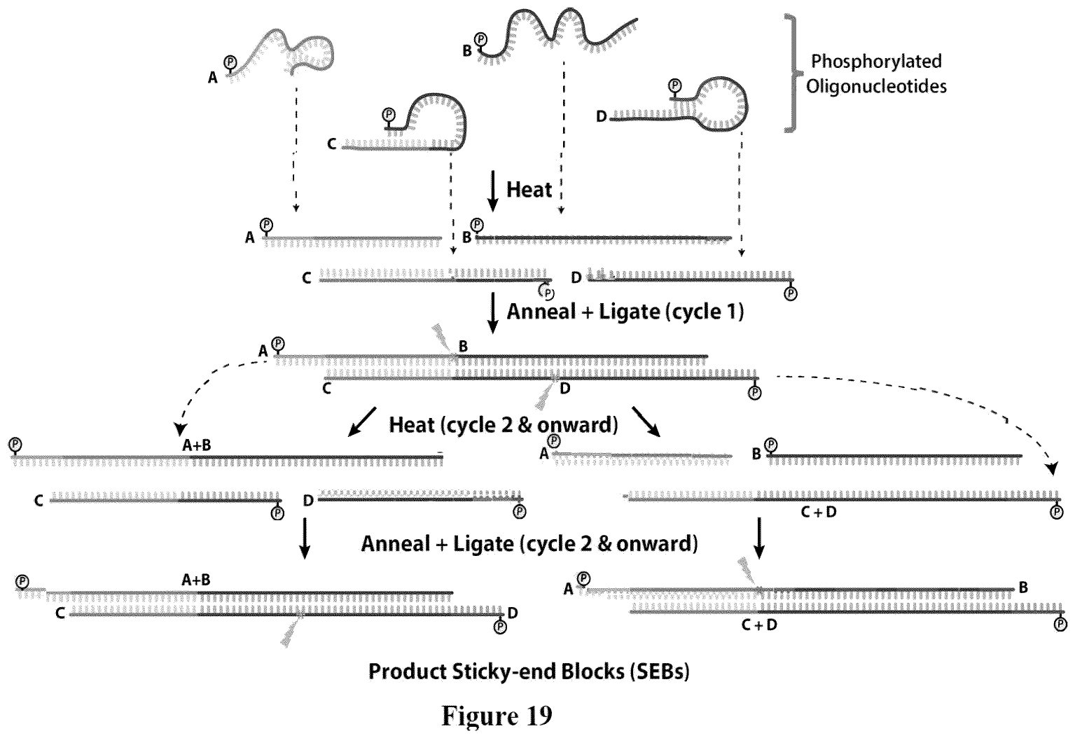

As shown in FIG. 19, the DNA precursors in the HLTA or CHTLA reaction are comprised entirely of 5′ phosphorylated single-stranded DNA oligonucleotides. In this embodiment, the melting step serves to disrupt secondary structure within individual oligonucleotides that forms due to self complementarity. Note that in this embodiment, even when HTLA is performed for multiple cycles, the product can be comprised entirely of an SEB, i.e., no blunt ended product forms. SEBs thus produced in separate reactions can then be joined by typical ligation to form a larger assembly. Alternatively, the end product can be designed to be a cccDNA. Specifically, seven single-stranded DNA oligonucleotides were designed to generate using CHTLA a 248 base-long SEB wherein 240 Watson-Crick base pairs were formed and 4-base sticky ends were present.

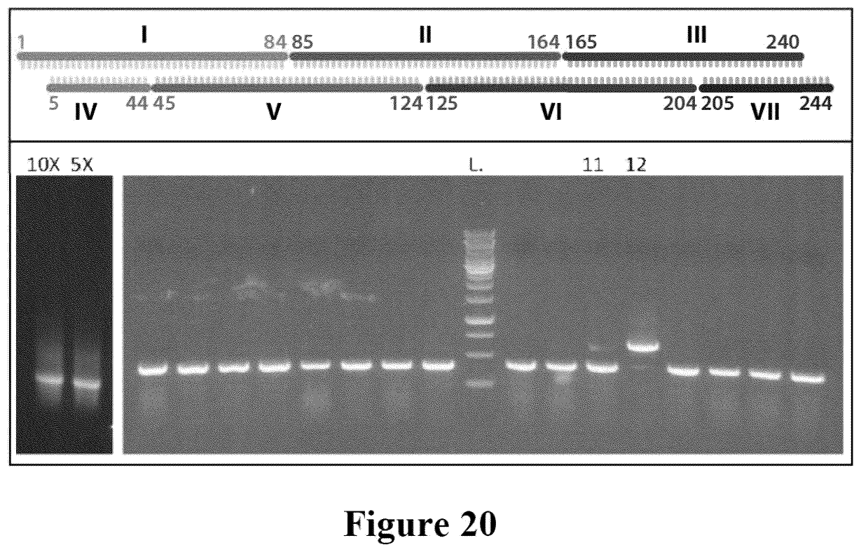

As shown in FIG. 20, the resultant CHTLA products were conventionally ligated to a restriction enzyme-digested vector plasmid and subsequently transformed into E. coli. Colony PCR of resultant clones demonstrated that 15/16 carried a plasmid insert for the expected 248 base length, and Sanger DNA sequencing of one clone demonstrated that the insert was of the correct DNA sequence.

That which is claimed is:

1. A method of forming Sticky-End Blocks (SEB) with 5′ or 3′ overhangs, the method comprising:
providing at least three precursor single-stranded, double-stranded DNA fragments or an admixture thereof and designed to correctly assemble to generate a defined DNA sequence;
introducing the at least three precursor DNA fragments into a buffer medium;
applying heat at a melting temperature to cause melting of the at least three precursor DNA fragments; and
lowering the melting temperature for annealing in the presence of a thermostable ligase, thereby generating a heteroduplex double-stranded DNA formed by base pairing of complementary regions juxtaposed on DNA ends for further ligation thereby generating a new larger double-stranded DNA product with single stranded ends with 5′ or 3′ overhangs to form the sticky-end blocks (SEB) or wherein the 5′ or 3′ overhangs are complementary, a covalently closed circular DNA is formed.

2. The method of claim 1, wherein the at a pH of the buffer medium is about 6.0 to about 9.

3. The method of claim 1 wherein the buffer medium comprises Tris-HCL, MgCl₂, KCl, NAD, DTT, and Triton X-100.

4. The method of claim 1, wherein the temperature for melting is determined by the size of the sequence and in a range from about 37° C. to 100° C.

5. The method of claim 1, wherein the heating time frame for melting ranging from 30 seconds to 10 minutes.

6. The method of claim 1, wherein annealing is conducted at a temperature from 5° C. to 40° C. lower than the melting temperature.

7. The method of claim 1, wherein the time frame for annealing is from about 4 mins to 10 mins.

8. The method of claim 1, wherein the size precursor DNA fragments range from about 20 nucleotides to thousands of nucleotides in length and can be single or double stranded.

9. The method of claim 1, wherein the buffer medium comprising Tris-HCL, MgCl₂, KCl, NAD, DTT, and Triton X-100, at a pH from about 7.5 to about 8, with a melting temperature from about above 80° C. for about to 3 minutes, wherein the annealing temperature is about 10° C. to 20° C. lower than the melting temperature and for about 4 mins to 6 mins, wherein the steps of melting, annealing and ligation are conducted multiple times range from 2 to 12 times to produce blunt-ended products.

10. The method of claim 1, wherein the steps of melting, annealing and ligation are conducted multiple times range from 2 to 30 times to produce sticky-ended products.

11. The method of claim 9 wherein the sticky-ended sequences can be ligated with a T4 ligase to provide longer and extended sequences.

12. The method of claim 9, wherein the size of Precursor DNA fragments range from about 20 nucleotides to thousands of nucleotides in length and can be single or double stranded.

13. The method of claim 9 wherein temperature related to melting, annealing and ligation ranges from about 95° C. to a lower temperature of about 60° C. for one, two, three, or more cycles.

14. The method of claim 1, wherein the defined sequence of DNA comprises one or more random or variable nucleotides.

15. A method of forming a mixture of sticky-ended heteroduplex DNAs and blunt-ended DNA products, the method comprising:
providing at least three precursor single-stranded, double-stranded DNA fragments or an admixture thereof and designed to correctly assemble to generate a defined DNA sequence;
introducing the at least three precursor DNA fragments into a buffer medium at a pH of about 7.5 to about 9 and comprising Tris-HCL, MgCl₂, KCl, NAD, DTT, and Triton X-100;
applying heat at a temperature to cause melting of the at least three precursor DNA fragments, wherein the temperature for melting is determined by the size of the sequence and can range from about 60° C. to 100° C. with a heating time frame ranging from 30 seconds to 10 minutes;
lowering the temperature for annealing in the presence of a thermostable ligase, wherein annealing is conducted at a temperature from 5° C. to 40° C. lower than the temperature for melting and for a time frame ranging from 4 mins to 10 mins thereby generating heteroduplex double-stranded DNA formed by base pairing of complementary regions with single stranded ends and thereby forming the sticky-end blocks with 5′ or 3′ overhangs; and
repeating the step of heating and annealing steps multiple times to form an admixture of SEBs and blunt-ended DNA sequences.

16. The method of claim 15, wherein the sticky-ended complimentary DNA sequences are further ligated by using a DNA ligase capable of ligating sticky ends and forming extended nucleotide sequences and/or circular DNA.
